(19) United States
(12) Patent Application Publication
(10) Pub. No.: US 2007/0079833 A1
(43) Pub. Date: Apr. 12, 2007

(54) Intraoral mandibular advancement device for treatment of sleep disorders, including snoring, obstructive sleep apnea, and gastroesophageal reflux disease and method for delivering the same

(76) Inventor: Steven B. Lamberg, Northport, NY (US)

Correspondence Address: Mitchell A. Stein, Esq.;STEIN LAW, P.C., 24 Woodbine Avenue, Suite 4, Northport, NY, 11768, US

(21) Appl. No.: 11/416,991

(22) Filed: May 3, 2006

Related U.S. Application Data
(60) Provisional application No. 60/724,597, filed on Oct. 7, 2005.

(51) Int. Cl. A61F 5/56

(52) U.S. Cl. 128/848

(57) ABSTRACT

An intraoral mandibular advancement device to treat problems associated with sleep disorders in a user having an obstructed airway, the disorders including, without limitation, snoring, obstructive sleep apnea, gastroesophageal reflux disease and/or bruxism having a main body for attachment to the user's mouth and having a central portion; a protrusive element distending from the central portion of the main body such that when worn by the user the element causes mandibular advancement sufficient to expand the oropharangeal space and reduce the obstruction; and a retainer extending from the main body for retention of the device in the user's mouth when worn during the user's sleep state. The main body is either complimentary with the user's palate and lingual surfaces of the maxillary teeth, rests against the palate and lingual surface of the user's anterior mandibular teeth when the device is inserted in the user's mouth, or customized to rest against two, four or six of the user's front teeth. The protrusive element is of distension between 5 and 15 mm, and optimally 10 mm downwardly from the main body, and can be adjusted such that the advancement ranges between 1.0 and 7.0 mm, and having an initial treatment protrusion of 3.0 to 4.0 mm, or ½ of the total potential maximum protrusive range. A method for diagnosis and delivery of the device is also shown.

### CLAIM OF PRIORITY

The instant application claims priority from provisional application, U.S. Ser. No. 60/724,597, dated Oct. 7, 2005 the contents of which are incorporated by reference.

### FIELD OF THE INVENTION

The present invention relates generally to devices for treatment of sleep disorders, and more particularly to an intraoral mandibular advancement device for the treatment of snoring, obstructive sleep apnea, gastroesophageal reflux disease, and bruxism and method for delivering the same.

### BACKGROUND OF THE INVENTION

Among the health problems associated with sleep disorders are inter alia, snoring, gastroesophageal reflux disease (“GERD”), obstructive sleep apnea (“OSA”), bruxism, diabetes, high blood pressure (hypertension), and increased risk of stroke and/or heart attack.

The most widespread sleep disorder is snoring which is a common affliction that affects tens of millions of people worldwide, and can potentially have very serious health and social consequences. Snoring is a sound produced by the vibration of tissue caused by a breathing obstruction during sleep. There are many factors associated with snoring including, but not limited to: heredity, body weight, age, gender, smoking history, nasal, tonsil, or soft palate deformities, use of alcohol, allergies, and sleep position. Commonly, both the snorer and the snorer's sleep partner lose sleep due to the snorer's snoring. The sleep partner is awakened by the sounds caused by snoring, perhaps repeatedly each night. Movement by the sleep partner in subsequently attempting to fall back asleep may then awaken the snorer, if the noise of his or her own snoring has not already done so. Lack of sleep subsequently leads to daytime fatigue, a compromised immune system, poor mental and emotional health, irritability and lack of productivity, which can lead to further repercussions. Snoring is thus problematic for many people.

Obstructive Sleep Apnea (“OSA”) is a potentially lethal sleep and breathing disorder defmed as the cessation of breathing for 10 seconds or more (an apnea) at least five times per hour of sleep. In severe cases, individuals can wake up 300 times per night. When breathing is interrupted by an obstruction in the airway, the body reacts by waking enough to start breathing again. Arousals may occur hundreds of time each night, and may not fully awaken the individual, who remains otherwise unaware of the loud snoring, choking and gasping for air typically associated with OSA.

Although not all snorers have OSA, snoring is a cardinal symptom of OSA and clearly indicative of and associated therewith. Many OSA sufferers do not receive a sufficient amount of sleep due to repeated apneatic events and arousals which act to prevent REM and deep stage sleep, which can lead to chronic daytime exhaustion and long-term cardiovascular stress. OSA is the direct cause of at least 38,000 deaths each year. An estimated 30 million Americans suffer from some degree of OSA, yet less than 3% are currently undergoing any type of treatment.

Pathogenesis of OSA involves a combination of reduced upper airway size and altered upper airway muscle activity, which causes oral tissue to collapse, and hence a blockage to occur. Other factors which are thought to contribute to OSA include tongue size, soft palate volume, a retrognathic mandible, an anteroposterior discrepancy between the maxilla and the mandible, and obesity.

Snoring and OSA are often associated as generally both are caused by blockage of the pharyngeal airway by, for example, excess tissue when various muscles of the body, including the tongue, relax. As the tongue relaxes, it moves posteriorly, blocking the breathing airway. When the breathing airway is blocked, exhaled air is forced through the airway with increased velocity thereby causing vibration of the tongue, tissue, or other obstruction, thereby creating noise. Snoring is caused by the partial obstruction of breathing during sleep while OSA occurs when the tongue and soft palate collapse onto the back of the throat and completely block the pharyngeal airway, thereby stopping breathing during sleep and restricting the flow of essential oxygen. Thus, a correlation between snoring and OSA is generally recognized in the medical community.

Many attempts have been made to reduce or eliminate snoring and OSA in individuals. Various types of surgery, including tracheostomy, surgery of the soft palate and oropharynx, and reconstructive surgery have been utilized in the treatment of snoring and OSA. Invasive surgery however is costly and not without risk, which effectively eliminates this modality as a truly viable solution to snoring and OSA for the general population. Indeed, many practitioners and patients alike would generally seek to avoid surgical intervention and would welcome a minimally invasive route—a general object and feature of the instant invention.

It has been recognized that opening or enlarging an airway that may be constricted due to collapse of tissue about the oropharynx will normalize the airflow to the lungs and in doing so will have a direct effect of resolving snoring and OSA as well as diminishing pressure fluctuations in the esophagus which have been associated with GERD. Known in the art are devices (heretofore cumbersome or ineffective) which are designed to restrict the velocity of the ingress and egress of air with the goal of reducing or eliminating snoring. It is scientifically concluded herein that by opening the aperture of the airway and maintaining the same, the consequences of a collapsed airway are avoided. When the airway is collapsed, the velocity of air flow increases and causes the vibration of the tongue, soft palate, and other tissue present in the pharyngeal wall during sleep. Thus, when expanded and maintained by the invention herein, the airflow is also maintained and snoring and OSA (where it theretofore resulted therefrom) are minimized to eliminated.

For example, U.S. pat. No. 6,263,877 to Gall shows a device which essentially consists of a mouth guard with a small aperture for which air can pass through. The amount of air flow is therefore directly proportional to the size of the aperture. U.S. pat. No. 15,642,738 to Lilly, Jr. shows a device which is designed to eliminate snoring via a membrane which is placed on the exterior surfaced of the anterior teeth to reduce the volume of air which is allowed to flow in and out of the mouth. U.S. pat. No. 4,817,636 to Woods likewise shows a device which seeks to eliminate snoring via a membrane which is placed on the exterior surfaced of the anterior teeth to reduce the volume of air which is allowed to flow in and out of the mouth. Yet, reduction of airflow can cause a cascade of other symptomology and situations, including potential OSA.

Also known in the art are devices which seek to reduce or eliminate snoring and/or OSA by inhibiting vibration of the soft palate. For example, U.S. pat. No. 6,467,485 to Schmidt shows a device made of a flexible material which is placed over the soft palate in an attempt to inhibit vibration of the soft palate. U.S. pat. No. 4,669,459 to Spiewak shows an intraoral device which seeks to eliminate snoring by positioning a button on the soft palate to prevent vibration thereof. Yet, such devices do not address the source of the problem or intervene to increase the airway (as does the instant invention), and thus the cascade effect is still a present risk if such devices are employed.

Various devices have sought to alleviate snoring by aiming to keep the pharyngeal airway open to prevent the collapse of the tongue and soft tissues in the back of the throat, yet are bulky and uncomfortable, often resulting in reduced patient compliance. For example, U.S. pat. No. 6,386,201 to Fard shows a pillow which seeks to keep the user's head in proper position to make snoring less likely. U.S. pat. No. 4,366,815 to Broomes and U.S. pat. No. 5,357,981 encompass other designs aimed at maintaining the user's body in a position which makes snoring less likely. U.S. pat. No. 6,668,834 shows a device which seeks to eliminate or reduce snoring by maintaining the user's chin above the user's chest while in the supine position in order to facilitate breathing as well as to aid in the prevention of snoring.

Positioning of the body does not eliminate the cause of snoring. Since tongue posture appears to have a substantial effect on upper airway morphology, several devices show a design which seeks to hold the tongue in a forward position in order to increase the diameter of the pharangeal airway during sleep. For example, U.S. pat. No. 6,494,209 to Kulick shows a device which consists essentially of a mouthpiece with an anterior suction mechanism which creates a vacuum which acts to maintain the tongue in an anterior position. U.S. pat. No. 6,408,851 shows a device which clamps the tongue in order to maintain the tongue in a forward position. U.S. pat. No. 6,055,986 to Meade shows a device for the reduction of snoring which encompasses both upper and lower portions which together form a cavity which encompasses the user's tongue when in use to maintain the tongue in a predetermined position and/or to prevent the tongue from moving posteriorly and thus blocking the pharangeal airway. U.S. pat. No. 5,988,170 shows a snoring prevention apparatus consisting essentially of a mask having a tongue depressing member, which acts to maintain the tongue in an anterior position during use. U.S. pat. No. 6,845,774 to Gaskell shows a mandibular splint which acts to maintain the user's jaws at a predetermined space apart to prevent the tongue from moving posteriorly in order to open the breathing passage. One can only imagine how truly uncomfortable such devices must be if used at all, and thus the likelihood of patient compliance is small to nil.

There is also recognition by those of ordinary skill in the art of a correlation between OSA and nocturnal gastroesophaegal reflux disease (“GERD”). Both OSA and GERD involve a reduction in air pressure in the trachea and esophagus caused by blockage during sleeping, thereby resulting in an increase in the draw of pressure through the esophagus as the diaphragm distends. While the body intends to increase breathing by such distention, the blockage results in the pulling of fluid upwardly in the esophagus and hence results in GERD for the snorer or OSA patient. Yet, the currently recommended solutions involve some version of a “CPAP” (continuous positive airway pressure) or pharmacological intervention. The former involves a face mask and a pump to provide pressure directly to the trachea of the patient which is cumbersome and unaesthetic. The latter obviously includes a plethora of side effects associated with the pharmacology employed, while not directly addressing the physical/mechanical source of the problem.

It is herein recognized that advancing the mandible in an anterior position relative to the maxilla during sleep opens the pharyngeal airway by indirectly urging the tongue forward to stimulate activity of the muscles in the tongue and thereby also increases the forward rigidity of the tongue. Since the tongue attaches to the posterior portion of the mandibular symphysis, advancing the mandible forward relative to the maxilla also pulls the tongue forward, thus preventing the tongue from obstructing the pharyngeal airway. Since the palatoglossus muscle attaches from the tongue to the soft palate, resultant forward movement of the tongue thereby creates tension on the soft palate thereby reducing vibration. Mandibular advancement devices therefore function to move the lower jaw, and hence the tongue forward to open the oropharynx. Snoring thus decreases proportionally with the increase in airway size or diameter.

It should therefore be appreciated that by so urging, and as an object of the instant invention, there can be a positive effect on reduction of snoring and OSA, and also upon GERD which is associated with the existence of large intrapleural pressure swings, which occur during apneatic events.

A number of different appliances for the treatment of snoring and/or OSA which move the mandible forward relative to the maxilla have been suggested, yet such devices typically fit over both the arches of teeth and are thus large and cumbersome. Several such different devices allegedly for the treatment of snoring and/or OSA move the mandible forward relative to the maxilla can be observed in the following U.S. patents: Halstrom, U.S. pat. No. 6,729,335; Thorton, U.S. pat. No. 6,516,805; Orrico, U.S. pat. No. 6,170,485; Belfer, U.S. pat. No. 6,092,523; Bergersen, U.S. pat. No. 6,129,084; Fenton, U.S. pat. No. 5,499,633; Tomasi, U.S. pat. No. 5,313,960; Hays, U.S. pat. No. 5,277,202; Shapiro, U.S. pat. No. 5,177,816; and Strong, U.S. pat. No. 6,526,982. These devices consist essentially of acrylic or elastomeric upper and lower bite trays which fit over both the maxillary and mandibular teeth, respectively, with some connecting means to shift the user's bite so that mandible is urged forward relative to the maxilla. Thus, to achieve anterior movement of the mandible, these devices essentially move one jaw against the other in a cumbersome manner which no doubt has an effect on patient compliance.

Such appliances which fit over both arches of teeth were derived from the orthodontic domain where it has ordinarily been considered prudent to cover all the teeth in order to prevent undesired movement (eruption) of any teeth not covered by the device resulting from a lack of opposition. Studies have shown however that partial coverage of the teeth during sleep does not result in undesired eruption of uncovered teeth, therefore rendering previous concerns unwarranted. These devices are therefore unnecessarily bulky and difficult to fit over the user's teeth. Bulky devices also occupy a large portion of the oropharyngeal volume, thus making breathing, and the passage of air around the bulky device difficult —the very antithesis of the goal herein sought: to increase the dimensional size of the airway. Breathing difficulty substantially reduces the efficacy of such devices to treat snoring and/or OSA, let alone the secondary effects of GERD caused by airway obstruction (which are not even mentioned in connection with such devices).

Many devices claimed for treatment of snoring and/or OSA which move the mandible forward relative to the maxilla fit over both the arches of teeth and also move one jaw substantially against the other, thereby posing other potentially damaging effects. Mandibular advancement devices that fit over both the maxillary and mandibular teeth are typically held nearly stationary, thereby restricting movement, causing discomfort, and potential permanent repositioning of the jaw. Since these types of devices restrict the user's natural lateral movements as well as anterior and posterior movements, continued use can potentially aggravate the tempromandibular joint (TMJ) and the related facial musculature, which would worsen over time, with continued use. Thus, it is an object of the instant invention to permit such movements, increase comfort and compliance, and additionally avoid TMJ effects.

Also shown in the art are appliances which are worn only upon the user's maxillary teeth, thus leaving the mandibular teeth uncovered. For example, U.S. pat. No. 5,915,385 to Hakimi discloses a snore and stress relief device which seeks to engage the user's upper dentition and includes an anterior extension which appears to advance the user's lower jaw forward with respect to the user's upper jaw such that the user's upper airway is enlarged and the passage of air through the upper airway is facilitated. The device disclosed in Hakimi has a posterior portion which fits against the dorsal surface of the user's soft palate to secure the device, which may also cause gagging or other discomfort, thus reducing or eliminating efficacy.

Although prior devices may have effect in treating snoring and OSA by moving the mandible forward relative to the maxilla to open the airway, such devices have unwanted side effects and/or are ineffective due to, inter alia, their bulk. As stated herein, many of the devices which appear to be designed to alleviate snoring and OSA restrict the user's natural nocturnal movements of the lower jaw which can potentially aggravate the tempromandibular joint and related jaw muscles and ligaments. Due to their bulk, devices prior to that shown herein that claim a design to eliminate or reduce snoring and/or OSA rely largely on the user's ability to breath through the nose. Thus, such devices would not function properly with users who have difficulty breathing through their noses. Furthermore, many of these devices must be removed or unhinged or otherwise disengaged to allow the user freedom to speak, swallow, or drink water, thus creating serious inconvenience to the user (and poor aesthetics). It is thus an object of the instant invention to overcome these shortcomings in prior devices.

Gastro Esophageal reflux disease (“GERD”), also known as acid reflux, is a chronic condition which affects at least 5 to 7% of the global population, including over seven (7) million people (reportedly as high as 30 million) in the United States alone. GERD is characterized by movement of the liquid contents of the stomach is from the stomach and into the esophagus. Almost everyone experiences gastroesophageal reflux at some time or another. When reflux is frequent or severe enough to cause more significant problems, it is characterized as GERD. GERD can cause serious complications including inflammation of the esophagus from stomach acid that causes bleeding and/or ulcers. Asthma, chronic cough, and pulmonary fibrosis may be aggravated or even caused by GERD.

The liquid content of the stomach typically contains acid, pepsin, and bile, which are harmful to the delicate lining of the esophagus when regurgitated. Damage to the esophageal lining can result in scarring and narrowing of the esophagus, and can be linked to the development of esophageal cancer.

Pressure created by the lower esophageal sphincter (“LES”) and diaphragm surrounding the LES ordinarily create a barrier to reflux. During snoring or an apneatic event, however, a large negative intrapleural pressure swing often occurs facilitating reflux events. Thus, blockage of the esophageal airway creates a negative pressure which allows reflux to occur by facilitating the upward movement of the liquid contents of the stomach. When in an upright position, most refluxed liquid naturally flows back into the stomach due to the force of gravity. When in a supine position however, most refluxed liquid is forced upwardly, into the esophagus, typically causing damage thereto. Due to the increased potential for reflux during sleep in the supine position, it is therefore beneficial to have a method of treatment which can be applied during sleeping hours to maintain the pressure system without creating negative esophageal pressure and concomitant potential reflux. See, e.g., Green, BT, et al., Marked Improvement in Nocturnal Gastroesophageal Reflux in a Large Cohort of Patients with Obstructive Sleep Apnea with Continuous Positive Airway Pressure, Arch Intern. Med., 2003 Jan 13;163(1):41-5.

Symptoms of gastroesophageal reflux disease include regurgitation, heart burn, difficulty swallowing, chest pain and nausea. As a chronic disease, treatment of gastroesophogeal reflux disease typically continues indefinitely. It is therefore preferable that a method of treatment for gastroesophogeal reflux disease is comfortable for nightly use.

Among the heretofore shown methods used to treat GERD are the use of antacids, histamine antagonists, proton pump inhibitors, promobility drugs which stimulate the muscles of the gastrointestinal tract, foam barriers which form a physical barrier to the reflux of liquid, and surgery. For example, U.S. pat. No. 6,979,750 to Scanlan, et. al. shows thyronamine derivatives and analogs for the treatment of GERD. U.S. pat. No. 6,974,837 to Jerussi, et. al. shows pharmaceutical compositions that comprise a racemic or optically pure sibutramine metabolite and a phosphodiesterase inhibitor for the treatment of GERD. U.S. pat. No. 6,548,518 to Rubin, et. al. shows the use of norcisapride in combination with proton pump inhibitors or H2 receptor antagonists for the treatment of GERD.

Continuous Positive Airway Pressure (“CPAP”) therapy has been shown to reduce GERD by increasing intra-esophageal pressure. Yet these devices have drawbacks, as herein stated, and thus are not of choice especially if non-invasive, easily placeable and removable, and not uncomfortable alternatives, like that of the instant invention, are available. Although there exists a broad range of dentally retained intraoral appliances worn at night for the treatment of snoring and obstructive sleep apnea, no such appliances heretofore satisfied such criteria and, also, were directed at treatment of GERD. It is thus an object of the instant invention to present a noninvasive alternative to drug therapy and surgical intervention for the treatment of GERD.

Bruxism is the technical description for the clenching, gnashing or grinding of teeth. Bruxing is common in almost all individuals and can occur subconsciously or when an individual becomes stressed or aggravated. The risk of bruxim increases with age, stress, and use of caffeine, nicotine and other drugs. In most cases, bruxism is mild and does not require treatment. When bruxing is more frequent however it can lead to jaw disorders, headaches, earaches, damaged teeth, chronic facial pains, and other problems. Therapy such as muscle relaxers and botox are currently used as remedies for bruxism. Alleged dental solutions include mouth guards and splints which appear to be designed to prevent damage to the teeth and/or decrease muscle activity. For example, U.S. Pat. No. 7,004,172 to Zacco shows a device which seeks to separate a user's mandibular and maxillary teeth by creating a space in the posterior portion of said mandibular and maxillary teeth. U.S. Pat. No. 6,886,566 to Eubank shows a device having a multiplicity of arches which act to maintain the tempromandibular joint in the proper position in an attempt to prevent bruxing. U.S. Pat. No. 6,675,804 to Pivovarov shows a device which maintains the tongue and mandibular and maxillary teeth in a position which seeks to eliminate the potential for bruxing. However, the device in Pivovarov encompasses both the mandibular and maxillary teeth, and is bulky and uncomfortable. U.S. Pat. No. 6,604 to Duncan shows a device that contains guide elements to position the device relative to the mouth and teeth and at the same time appears to prevent mating of the teeth when the mouth is closed and therefore gnashing or grinding of teeth during sleep. Yet, such devices heretofore shown in the art that are aimed to prevent bruxism or damage caused by bruxing fail to address and/or effectively aid in the treatment of snoring, OSA and GERD. An object and feature of the instant invention is the separation of the posterior teeth thereby eliminating the possibility of clenching which is a prerequisite for bruxing, while nonetheless permitting nocturnal jaw movements.

Hence, heretofore unknown, and an object of the instant invention, is the presentation of an effective intraoral appliance for treatment of sleep disorders including snoring, OSA, GERD and/or bruxing by moving the user's mandible forward with respect to the user's maxilla in order to open the airway in a manner which is comfortable, easily placeable, and without adverse effects. As shown above, prior devices have struggled to optimize the size and design parameters to facilitate the dual goals of comfort and effectiveness, but have woefully failed to achieve this goal. Thus, compliance is a predominant issue.

### SUMMARY OF THE INVENTION

In accordance with the teachings of the present invention, an intraoral mandibular advancement device is provided which substantially eliminates or reduces disadvantages associated with prior devices in connection with sleep disorders. More specifically, it is a feature of the instant invention to provide an intraoral mandibular advancement device for the treatment of sleep disorders, primarily snoring, and also including OSA, GERD and/or bruxism by, inter alia, urging the user's lower jaw forward via a protrusive element which pushes against the lingual surfaces of the anterior mandibular teeth, all as more fully shown herein.

In particular, an intraoral mandibular advancement device is shown to treat problems associated with sleep disorders in a user having an obstructed oropharyngeal space, the disorders including, without limitation, snoring, obstructive sleep apnea, gastroesophageal reflux disease and/or bruxism having a main body for attachment to the user's mouth and having a central portion; a protrusive element distending from the central portion of the main body such that when worn by the user the element causes mandibular advancement sufficient to expand the oropharangeal space and reduce the obstruction; and a retention element extending from the main body for retention of the device in the user's mouth when worn during the user's sleep state. The main body is either complimentary with the user's palate and lingual surfaces of the anterior maxillary teeth, and extends downwardly against the lingual surfaces of the user's anterior mandibular teeth, or can be customized to rest against two, four or six of the user's front teeth.

The protrusive element is of distension between 5 and 15 mm, and optimally 10 mm downwardly from the main body, and can be adjusted anteriorly or posteriorly such that the advancement of the protrusive element relative to the main body ranges between 1 and 7 mm. The advancement of the protrusive element relative to the main body commences at a position approximately 3.0 to 4.0 mm or approximately ½ of the user's maximum protrusive range and is advanced, if required therefrom. A method for diagnosis and prescription of the device is also shown.

More particularly, an intraoral mandibular advancement device is provided which includes a main body comprised of materials providing the requisite strength and flexibility such as polymeric materials, acrylic materials, natural materials, rubbers, metals, silicones, vinyls, elastomeric materials, hard plastic, thermal plastic, and thermosensitive acrylic resin and combinations thereof. The preferred embodiment of the instant invention largely resembles an orthodontic retainer, yet encompasses additional elements in order to urge the user's lower jaw forward relative to the user's upper jaw for the elimination or reduction of sleep disorders including primarily snoring, as well as obstructive sleep apnea, gastroesophageal reflux disease. It is preferable that the lower jaw is advanced beginning at least 3.0-4.0 mm relative to the maximum intercuspated position (“MIP”) and moved anteriorly as required to achieve the goals stated herein. This 3.0-4.0 mm shift represents approximately 50% of an individual's maximum active protrusive range. The optimal degree of offset will vary with each user and hence adjustability is provided.

For the purpose of comfort, it is preferable that the lower jaw is not moved further anteriorly than necessary to gain patency in the airway. The instant invention includes a main body which is preferably complimentary with a user's palate and lingual surfaces of the maxillary teeth. The main body is thus essential horseshoe-shaped. Custom fitting with the user's dental arch ensures comfort while at the same time acting as a retention mechanism to secure the device in the user's mouth.

Extending from the central portion of the main body of the instant invention is a protrusive element which, while in use, rests behind the lingual surfaces of the user's anterior mandibular teeth in a manner to urge the lower jaw anteriorly. The length of the protrusive element is approximately 10.0 mm, yet the precise length will vary with the structure of mouth of each user, and is customized to facilitate comfort and efficacy. Anterior movement of the mandible by the instant device serves to urge the attached tongue forward as well as a result of the attachment, thereby achieving all of the aforementioned goals, objects and features. For example, said anterior movement of the mandible and tongue decreases the negative pressure in the esophagus and thereby reduces or eliminates GERD (when the same is caused thereby). Importantly, mandibular advancement, or protrusion, also creates more space behind the tongue and therefore increases the airway diameter in the oropharynx.

Said protrusive element further serves to position the mandible in place to inhibit the tongue from moving posteriorly during sleep in the supine position. When the instant inventive device is inserted in the user's mouth, the protrusive element fits snugly against the lingual surface of a plurality of the anterior mandibular teeth. When in use, the device is fixed between the user's maxillary arch and anterior mandibular teeth thus moving the mandible forward relative to the maxilla. As retention is a critical element in the design of any intraoral appliance, the device of the instant invention is designed to be retained in the user's mouth while withstanding the user's ordinary movements, which may include teeth grinding.

Said protrusive element, like the main body, can be comprised of materials known in the art which provide the requisite strength and flexibility such as polymeric materials, acrylic materials, natural materials, rubbers, metals, silicones, vinyls, elastomeric materials, hard plastic, thermal plastic, and thermosensitive acrylic resin and combinations thereof.

Retention of the device in the user's mouth during use is also achieved via a plurality of clasps which surround the exterior of the user's teeth. Said clasps are arranged at different positions of the device for optimal comfort and effectiveness. In one embodiment of the instant invention said clasps are posteriorly positioned over the upper left and right molars, on opposite sides of the mouth, and anteriorly positioned from cuspid to cuspid, on opposite sides of the mouth.

Retention of the device may also achieved by a retention arc formed by the main body in which the anterior mandibular teeth are fitted. The length of the retention arc may vary to encompass a plurality of teeth.

In one embodiment of the instant invention, said protrusive element is adjustable, in order to incrementally advance the degree of displacement between the main body and protrusive element. It is recognized that the smallest amount of mandibular advancement necessary to attain a patent airway should be used to maximize both efficacy and comfort. Yet, even in the non-adjustable version of the instant invention, material may be added or extracted to thereby modify dimensions to afford such maximization.

At least one embodiment of the instant invention can be fitted onto two, four or six anterior maxillary (upper) teeth while leaving mandibular teeth and the posterior maxillary teeth and remaining anterior maxillary teeth, if any, uncovered. Dispersing pressure over multiple teeth eliminates placing undue pressure on teeth while at the same time eliminating the potential that the teeth will be reoriented.

While in use, the instant invention is positioned on a varying number of anterior maxillary teeth thus preventing the posterior teeth to contact. Said contact on the anterior maxillary teeth, in addition to the lack of contact on the posterior teeth, prevents the elevator muscles from contracting thereby precluding grinding. Since the jaw muscles are prevented from contracting, bruxing (teeth grinding) is made less likely, thus rendering the instant invention effective in the treatment of bruxism.

Mandibular advancement effects the anatomy of the upper airway to allow increased air flow by, inter alia, (a) elevating the base of the tongue in resting position; (b) tensing the palatoglossus muscles and urging the soft palate (uvula) forward; (c) decompressing tissues around the pharynx and allowing the pharynx to expand; (d) stabilizing the lateral pharyngeal wall by applying tension to the pterygomandibular raphe, which is coupled to pharyngeal constrictors; and/or (e) splaying the tonsillar arches formed by the palatoglossus and the palatopharyngeal muscles, which leads to further stabilization of the lateral pharyngeal wall.

Due to its low bulk, the device of the instant invention is enclosed completely within the oral cavity of the user during use to permit a comfortable fit during use. Since the device fits completely within the oral cavity of the user, the device of the instant invention thereby permits adequate lip seal. Such an intraoral device as that shown herein permits the lips of the user to close thereby enjoying higher patient compliance and is also simpler for the user to adapt to using, thus creating less interference with sleep.

A principal feature and advantage of the instant invention is the provision of a device which is effective in the treatment of sleep disorders, primarily snoring, OSA, GERD and/or bruxism, as described.

A further feature of the instant invention is the provision of a device which advances a user's mandible relative to the user's maxilla.

A further feature of the instant invention is the provision of a device which minimizes the amount of materials used while advancing a user's mandible relative to the user's maxilla.

A further feature of the instant invention is the provision of a device which advances a user's mandible relative to the user's maxilla customized to fit a user's unique mouth structure.

A further feature of the instant invention is the provision of a device which opens a user's airway and prevents the same from subsequently becoming obstructed by the user's own oral tissue structure.

A further feature of the instant invention is the provision of a device which advances a user's mandible relative to the user's maxilla which is also removably maintained solely upon the user's upper dentition or palate.

A further feature of the instant invention is the provision of a device which advances a user's mandible relative to the user's maxilla which is also of low bulk.

A further feature of the instant invention is the provision of a device which advances a user's mandible relative to the user's maxilla which also allows the user to close his or her mouth so as to allow lip seal.

A further feature of the instant invention is the provision of a device which advances a user's mandible relative to the user's maxilla while also allowing adequate tongue space for comfort and facile movement of the tongue.

A further feature of the instant invention is the provision of a device which prevents posterior movement of the tongue by advancing the user's mandible relative to the user's maxilla to maintain the tongue in the proper position and to permit the unobstructed passage of inspiratory and expiratory air.

A further feature of the instant invention is the provision of a device which can be incrementally adjusted to vary the degree of anterior protrusion for optimal placement behind the user's anterior mandibular teeth.

A further feature of the instant invention is the provision of a device which advances a user's mandible relative to the user's maxilla without influencing occlusion (bite).

A further feature of the instant invention is the provision of a device which provides a comfortable fit for the user.

A further feature of the instant invention is the provision of a device which allows lateral movement of the mandible relative to the maxilla as well as anterior/posterior movement relative to the maxilla, for reasons of comfort.

A further feature of the instant invention is the provision of a device which allows the user freedom to speak, swallow, or drink water without removing, unhinging or otherwise disengaging the device.

A further feature of the instant invention is the provision of a device which precludes the contacting of posterior teeth by utilization of an anterior area of contact which thereby prevents clenching and bruxing.

The above description sets forth rather broadly the more important features of the present invention in order that the detailed description thereof that follows may be understood, and in order that the present contributions to the art may be better appreciated. Other objects and features of the instant invention will become apparent from the following descriptions considered in conjunction with the accompanying figures. It is to be understood, however, that the drawings are designed solely for the purposes of illustration and not as a definition of the limits of the invention, for which reference should be made to the appended claims.

### BRIEF DESCRIPTION OF THE DRAWINGS

In the drawings, wherein similar reference characters denote similar elements throughout the several views:

FIG. 1 is a perspective, environmental view of one embodiment of the intraoral mandibular advancement device of the instant invention shown during use in phantom.

FIG. 2 is a topographic view of one embodiment of the intraoral mandibular advancement device of the instant invention;

FIG. 3 is a bottom view of one embodiment of the intraoral mandibular advancement device of the instant invention;

FIG. 4 is a bottom view of one embodiment of the intraoral mandibular advancement device of the instant invention shown during use;

FIG. 5 is a cross-sectional view of one embodiment of the intraoral mandibular advancement device of the instant invention shown during use, as shown in FIG. 6;

FIG. 6 is a frontal perspective view of one embodiment of the intraoral mandibular advancement device of the instant invention;

FIG. 7 is a perspective view of one embodiment of the intraoral mandibular advancement device of the instant invention for attachment to six frontal teeth of the user;

FIG. 8 is a perspective view of the embodiment of the intraoral mandibular advancement device of the instant invention, as shown in FIG. 7, in use;

FIG. 9 is a perspective view of one embodiment of the intraoral mandibular advancement device of the instant invention for attachment to four frontal teeth of the user;

FIG. 10 is a perspective view of one embodiment of the intraoral mandibular advancement device of the instant invention, as shown in FIG. 9, in use;

FIG. 11 is a perspective view of one embodiment of the intraoral mandibular advancement device of the instant invention for attachment to two front teeth of the user;

FIG. 12 is a perspective view of one embodiment of the intraoral mandibular advancement device of the instant invention, as shown in FIG. 11, in use;

FIG. 13 is a vertical cross-sectional view of one embodiment of the intraoral mandibular advancement device of the instant invention;

FIG. 14 is a bottom view of the preferred embodiment of the intraoral mandibular advancement device of the instant invention showing adjustability as further defmed hereinbelow;

FIG. 15 is an enlarged, cross-sectional view of the adjustable mechanism of the device shown in FIG. 14;

FIG. 16 is a schematic, side elevational view of the preferred embodiment of the instant invention, shown in FIG. 14, inserted in the oral region of a user;

FIG. 17 is an enlarged, cross-sectional view of an alternative adjustable mechanism of the preferred embodiment of the intraoral mandibular advancement device of the instant invention;

FIG. 18 is a schematic, side elevational view of yet another alternative adjustable mechanism of the preferred embodiment of the instant invention shown in the oral region of a user;

FIG. 19 is a schematic, side elevational view of yet another alternative adjustable mechanism of the preferred embodiment of the instant invention shown in the oral region of a user; and

FIG. 20 is a flow chart of the underlying logic of the method and system of the preferred embodiment of the subject invention showing the sequence of events for making the same.

### DETAILED DESCRIPTION OF THE PREFERRED EMBODIMENTS

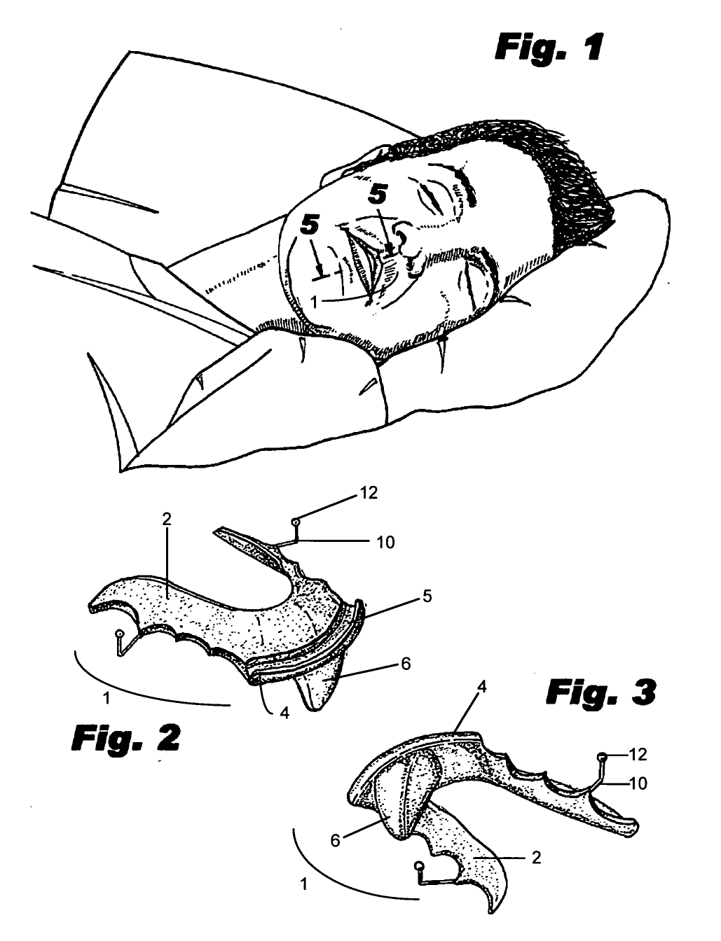

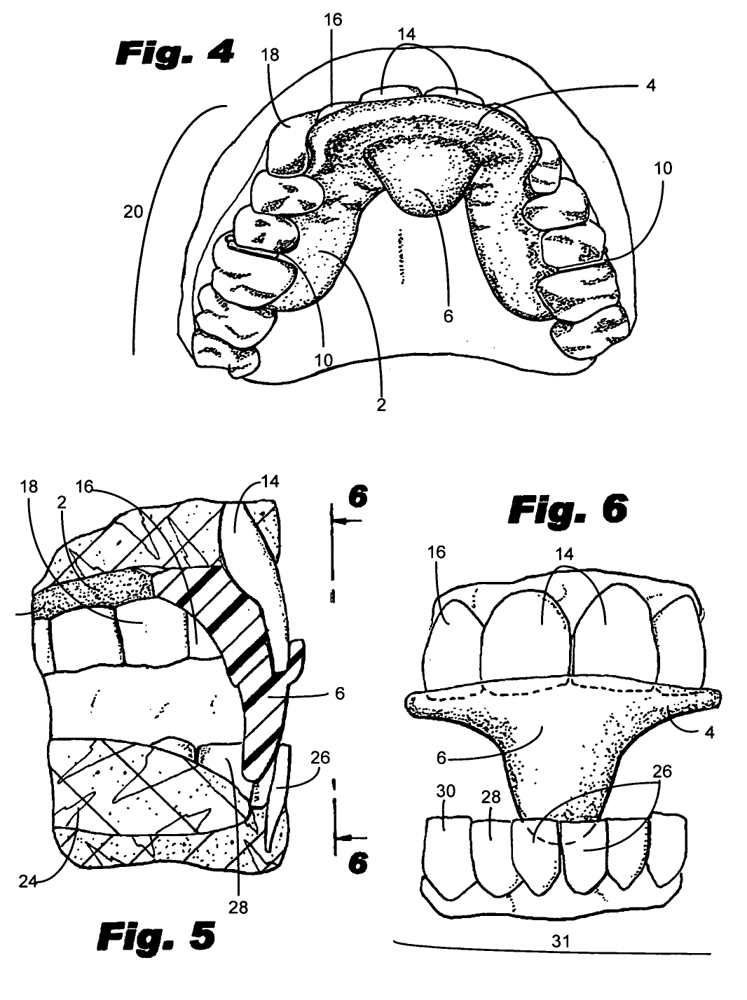

Shown in FIG. 1 is a perspective, environmental view of one embodiment of the intraoral mandibular advancement device of the instant invention shown worn by a typical person in the supine position. Device 1 permits the user to voluntarily open and close his or her mouth to speak or drink a glass of water, while also allowing for natural movement during sleep. As shown in FIGS. 2-6, intraoral mandibular advancement device 1 has a main body 2 which is customized to fit along the lingual surface of the user's maxillary teeth 20 and hard palate while leaving the soft palate and middle area of the palate uncovered during use. It should be appreciated by one of ordinary skill upon reading the text hereof and the figures herein, that the instant inventive device 1 disallows contact of the posterior teeth against each other thereby preventing contraction of the elevator muscles of the jaw and hence bruxism, as well as opening the airway as explained, thereby eliminating other sleep disorders including snoring, OSA and/or GERD.

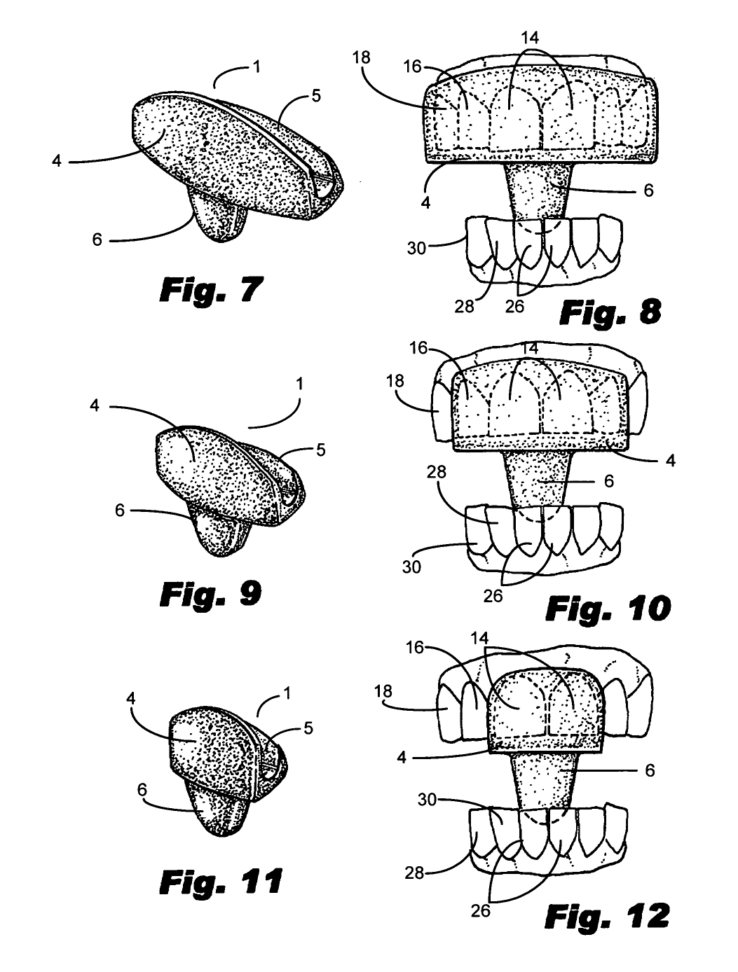

As shown in FIGS. 2 and 3, at the anterior end of main body 2 is retention arc 4 which is customized to fit a plurality of the user's anterior maxillary teeth for retention of the device in the user's mouth when worn during the user's sleep state. Retention arc 4 forms a trough 5 which, in this embodiment, conforms with the user's anterior maxillary teeth for positioning (and in FIGS. 7-12 serves to retain the device, as discussed further hereinbelow). Trough 5 formed by retention arc 4 is, in the embodiment shown in FIGS. 2-6, less than 1.0 mm in depth so that the incisal edges of the user's maxillary incisors 14 and 16 of the maxillary teeth 20 (FIG. 4) make contact with device 1 for location, while the remainder of device 1 provides for retention. As shown in FIG. 4, retention arc 4 covers only the user's maxillary central incisors 14, lateral incisors 16, thus leaving the user's remaining maxillary teeth 20, including maxillary canine teeth 18, uncovered.

Distending from the central portion of main base 2 is protrusive element 6. Protrusive element 6 is customized to conform with the lingual surface of the user's anterior mandibular teeth. Protrusive element 6 engages the lingual surface of the user's mandibular central incisors 26 (and, if compromised periodontally or biomechanically, then extended laterally to distribute the forces over mandibular lateral incisors 28) so as to maintain a forward posture of the user's mandible relative to the maxilla. As the mandible moves forward, so does attached tongue 24, thus maintaining tongue 24 in a forward posture. When worn by the user protrusive element 6 (when properly sized) thus causes mandibular advancement sufficient to expand the oropharyngeal space and reduce any obstruction therein, including the tongue 24.

Extending from the lateral anterior portions of main body 2 are clasps 10 for retention of the device in the user's mouth when worn during the user's sleep state. Clasps 10 are customized to cover the dental and exterior surfaces of the user's upper left and right molars, preferably the first molars. Clasps 10 can be made of surgical or stainless steel or other materials known in the art. At the distal end of clasps 10 are clasp ends 12 which fit adjacent to the exterior of the upper teeth gingival to the height of contour so as to engage the undercut in order to help retain the device in the user's mouth when worn during the user's sleep state. Clasps 10 and clasp ends 12 may comprise other retentive shapes without deviating from the letter, spirit of the instant invention as disclosed and claimed.

Shown in FIG. 6 is a frontal view of protrusive element 6 positioned in use between the user's maxillary teeth 20 and mandibular teeth 31. Retention arc 4 encompasses the user's central maxillary incisors 14 and lateral maxillary incisors 16, while leaving the user's remaining maxillary teeth, if any, uncovered. The distal end of protrusive element 6 fits adjacent to the lingual side of the user's central mandibular incisors so as to maintain a forward posture of the user's mandible relative to the maxilla while leaving the user's mandibular lateral incisors 28 and mandibular canine teeth 30 uncontacted.

Shown in FIG. 7-13 is an alternative embodiment of intraoral mandibular advancement device 1 in which the device consists essentially of a retention arc 4having a protrusive element 6. Retention arc 4 forms a trough 5 which encompasses the user's anterior maxillary teeth thereby retaining device 1 in the user's mouth during use.

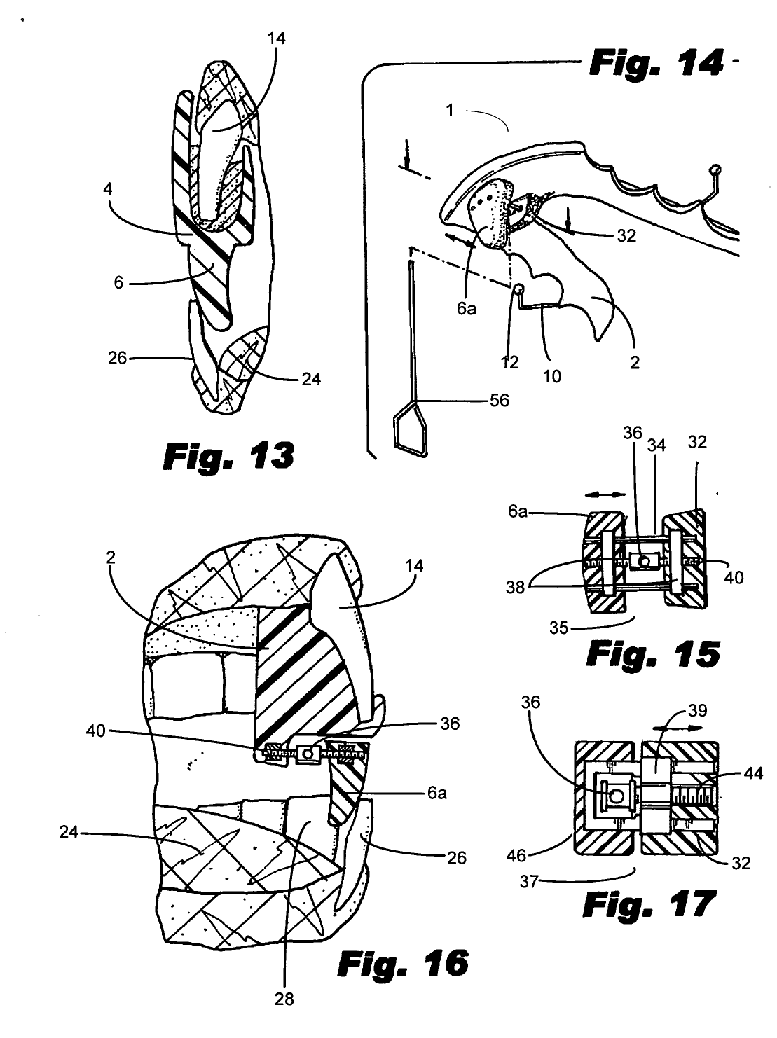

As shown in FIG. 8, FIG. 10, FIG. 12, and FIG. 13 protrusive element 6 engages the lingual surface of the user's anterior mandibular teeth 28 and 26 so as to maintain a forward posture of the user's mandible relative to the maxilla. As the mandible moves forward, so does the attached tongue 24, thus maintaining the tongue 24 in a forward posture. When worn by the user, protrusive element 6 thus causes mandibular advancement sufficient to expand the airway and reduce any obstruction therein, including the tongue 24.

As shown in FIG. 8, FIG. 10, and FIG. 12 protrusive element 6 contacts only the lingual surface of the user's central mandibular incisors 26, thus leaving the lateral mandibular incisors 28 and mandibular canine teeth 30 uncovered.

As shown in FIG. 8, the user's central maxillary incisors 14, lateral maxillary incisors 16, and maxillary canine teeth 18 are encompassed by device 1 to provide retention in the user's mouth during use; the user's remaining maxillary teeth, if any, remain uncovered by device 1.

As shown in FIG. 10, the user's central maxillary incisors 14 and lateral maxillary incisors 16 are encompassed by device 1 to provide retention in the user's mouth during use; the user's remaining maxillary teeth, if any, including the maxillary canine teeth, remain uncovered by the device 1.

As shown in FIG. 12, the user's central maxillary incisors 14 are encompassed by device 1 to provide retention in the user's mouth during use; the user's remaining maxillary teeth, if any, including the user's lateral maxillary incisors 16 and maxillary canine teeth 18 remain uncovered by the device 1.

Thus, six maxillary teeth are encompassed in FIGS. 7 and 8, four maxillary teeth in FIGS. 9 and 10, and two maxillary teeth in FIGS. 11 and 12.

Shown in FIG. 14-16 is an alternative embodiment of the intraoral mandibular advancement device 1 which is essentially similar to that disclosed in FIG. 2-5, yet contains an adjustable protrusive element 6a. Adjustable protrusive element 6a allows the user (typically via the dentist) to incrementally advance the degree of anterior protrusion for optimal placement behind the user's anterior mandibular teeth. It is recognized that the smallest amount of mandibular advancement necessary to attain a patent airway should be used to maximize both efficacy and comfort, and hence compliance.

The portion of main body 2 adjacent to expansion means 35 is expansion base 32. Also running perpendicular through adjustable component plates 38 is threaded rod 40 which can be rotated clockwise or counterclockwise to increase and decrease the amount of separation between adjustable protrusive element 6a and main body 2. Adjustable protrusive element 6a is attached to main body 2 by expansion means 35. Expansion means 35 contains a plurality of adjustment component plates 38 embedded in both adjustable protrusive element 6a and expansion base 32, which is continuous with main body 2. Adjustment component plates 38 have running perpendicular therethrough a plurality of parallel guiding rods 34, thereby acting to separate adjustable protrusive element 6a from main body 2. Because main body 2 is fixed in the user's mouth, increasing the amount of space between protrusive element 6a and main body 2 acts to urge the user's mandible forward relative to the user's maxilla so as to enlarge the volume of the user's airway or otherwise reduce its collapsibility. The user (typically via the dentist) may adjust the amount of mandibular advancement by inserting adjustment key 56 into aperture 36 and rotating clockwise or counterclockwise, depending on whether the user wishes expansion or contraction of protrusive element 6a relative to main body 2, and hence advance or withdraw the user's mandible relative to the user's maxilla.

Shown in FIG. 17 is a cross sectional view of an alternative adjustment mechanism 37 shown in a fully tightened mode. As adjustment is made via 36, screw 44 is advanced via nut 39 thereby separating portion 46 from portion 42, and the entire mechanism is thus urged outwardly causing advancement, in this embodiment. Mechanism 37 can be inserted in either direction for functionality.

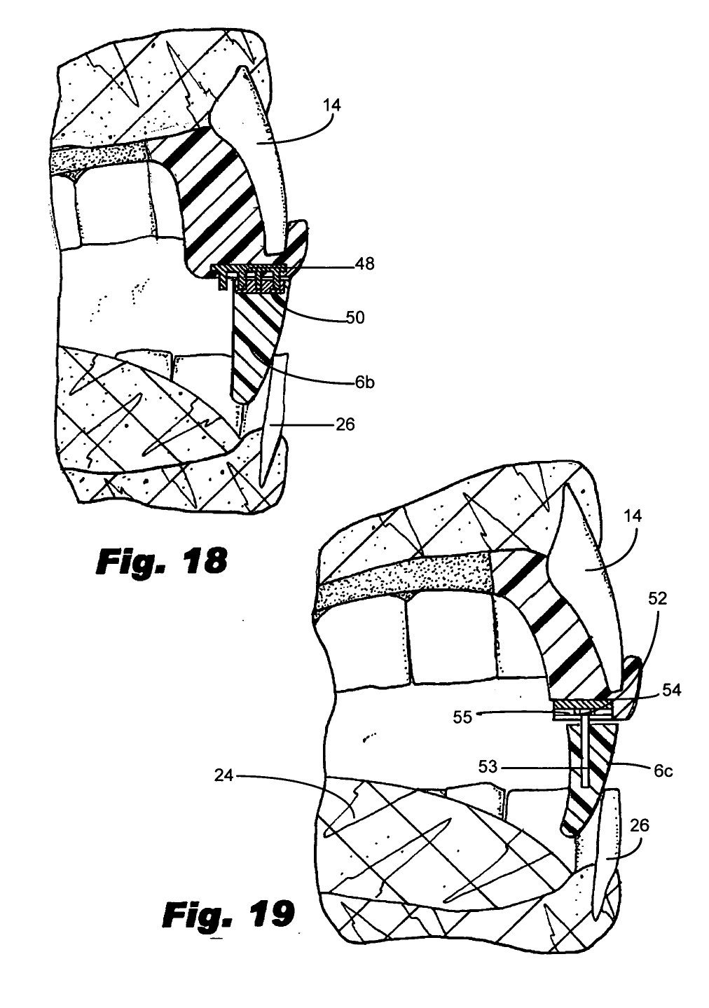

FIG. 18 is a schematic, side elevational view of one embodiment of the instant invention shown in the oral region of a typical person. In this embodiment upper adjustable snap assembly 48 engages lower adjustable snap assembly 50 such that each movement anteriorly or posterially causes protrusive element 6b to move accordingly. By snapping the device is thereby held in place. In this instance, about seven positions are available, although the assembly is not thereby restricted by one of ordinary skill in the art with the eye towards optimizing proper and sufficient displacement to open the airway.

FIG. 19 is a schematic, side elevational view of one embodiment of the instant invention shown in the oral cavity of a typical person. In this embodiment, rod 53 is contained in protrusive element 6c and is threaded in the upper portion thereof to engage sliding nut 52. Sliding nut 52 travels longitudinally along track 55 and is screwable to enable rod 53 and thereby protrusive element 6c to be adjusted and fixed in a desired location.

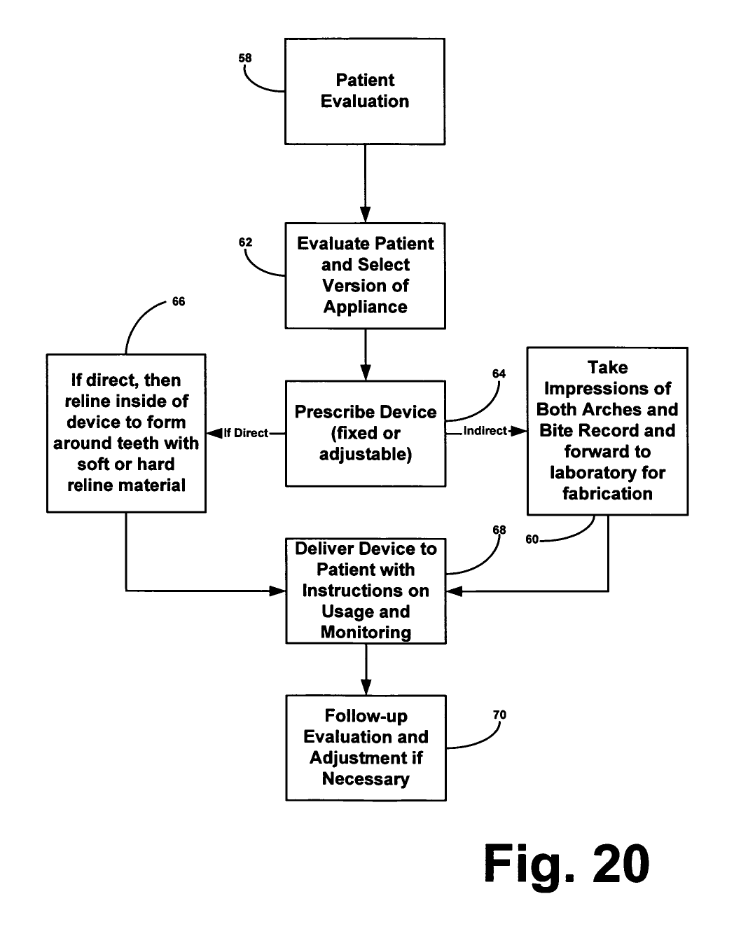

FIG. 20 is a flow chart of the underlying logic of the method and system of the preferred embodiment of the subject invention showing the sequence of events for making the same. In this instant, the method is shown wherein the patient is evaluated at step 58, and then evaluated and a version of the device is selected at step 62. The selected version of the device (whether tooth-borne or palate-borne) is further determined to be delivered as either a fixed appliance or made adjustable at step 64. If an adjustable version is selected, then the mechanism for adjustment is chosen from the plurality of mechanisms shown, or their like kind. At this point, one of two alternatives is presented. If the “direct” approach is selected, then the device is ordered, received, and must be customized for the patient by relining the inside of the device to form around teeth with soft or hard reline material at step 66. If the “indirect” approach is selected, then impressions are taken at step 60, and sent to the laboratory for fabrication of the selected device. The device, upon receipt is then delivered to the patient at step 68 with instructions on usage and monitoring. It is only through patient feedback (or that of the bed partner) that the precise dimensions can be fine tuned to ensure maximum relief from sleep disorders with minimum displacement of the mandible relative to the maxilla—the goal of the instant invention. Thus, there is follow-up and evaluation and adjustment if necessary via step 70. It should be appreciated by one of ordinary skill in the art upon reading and studying the instant invention that where such adjustment is to be made to a “fixed” or non-adjustable device, this occurs by the addition or subtraction of material from the protrusive element. Where the device provides for adjustment in any of a number of ways, then such adjustment is accomplished in that manner in accordance with the design selected.

The instant invention is shown to be effective, as demonstrated by the following studies:

Study 1

Patient of age 51 presents with loud snoring and moderate to severe apnea, theretofore treated with a CPAP device. The CPAP device is replaced with a fixed device in accordance with the subject invention creating a 4.0 mm anterior displacement of the mandible. Patient reports complete elimination of snoring while wearing the appliance for a 14 day trial.

Study 2

Patient of age 76 suffered from chronic snoring which was loud enough to awaken the patient's bed partner, theretofore treated with a number of pharmaceuticals. The pharmaceuticals were replaced with a fixed device in accordance with the subject invention creating a 4.0 mm anterior displacement of the mandible. Patient reports a significant reduction in snoring while wearing the appliance for a 14 day trial.

Study 3

Patient of age 50 suffered from snoring for many years, theretofore treated with pharmaceuticals such as Lipitor, as well as over-the-counter remedies such as vitamins and Tylenol. The pharmaceuticals and over-the-counter remedies were replaced with a fixed device in accordance with the subject invention creating a 4.0 mm anterior displacement of the mandible. Patient reports a significant reduction in snoring while wearing the appliance for a 14 day trial.

Study 4

Patient of age 55 suffered from snoring, theretofore treated with pharmaceuticals. The pharmaceuticals were replaced with a fixed device in accordance with the subject invention creating a 4.0 mm anterior displacement of the mandible. Patient reports a significant reduction in snoring while wearing the appliance for a 14 day trial.

Study 5

Patient of age 51 suffered from snoring which was loud enough to awaken people sleeping in adjacent rooms, theretofore untreated. A fixed device in accordance with the subject invention creating a 4.0 mm anterior displacement of the mandible was fitted for the patient. Patient reports a complete elimination in snoring while wearing the appliance for a 14 day trial.

Likewise, it is scientifically reasonable to conclude that such patients suffered a lowering of apneatic events and therefore GERD to the extent thereby related.

While there have shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the invention, therefore, to be limited only as indicated by the scope of the claims appended hereto.

I claim:

1. An intraoral mandibular advancement device to treat problems associated with sleep disorders in a user having an obstructed orophangeal space, the disorders including, without limitation, snoring, obstructive sleep apnea, gastroesophageal reflux disease and/or bruxism comprising:
(a) main body means for attachment to the user's mouth and having a central portion;
(b) a protrusive element distending from the central portion of said main body such that when worn by the user said element causes mandibular advancement sufficient to expand the oropharyngeal space, to open the airway and reduce the obstruction; and
(c) retention means extending from said main body for retention of the device in the user's mouth when worn during the user's sleep state.

2. The intraoral device of claim 1, wherein the main body is complimentary with the user's palate and lingual surfaces of the maxillary teeth.

3. The intraoral device of claim 1, wherein the protrusive element is customized to rest against the lingual surface of the user's anterior mandibular teeth when the device is inserted in the user's mouth.

4. The intraoral device of claim 1, wherein the main body is customized to rest against the user' central maxillary incisors, thus encompassing two (2) teeth.

5. The intraoral device of claim 1, wherein the main body is customized to rest against the user' central and lateral maxillary incisors, thus encompassing four (4) teeth.

6. The intraoral device of claim 1, wherein the main body is customized to rest against the user' central and lateral maxillary incisors and maxillary canine teeth, thus encompassing six (6) teeth.

7. The intraoral device of claim 1, wherein the main body is designed to rest on the user's palate, thus leaving the user's maxillary teeth, with the exception of the central and lateral incisors, and all mandibular teeth uncovered.

8. The intraoral device of claim 1, wherein the protrusive element is of sufficient dimension such that it causes an elevation of the base of the user's tongue thereby bringing the tongue forward.

9. The intraoral device of claim 1, wherein the protrusive element is of sufficient protusion to cause a pulling of the user's palotoglossus muscles and thereby applying tension on the tissues of the soft palate thereby reducing vibratory noises associated with snoring.

10. The intraoral device of claim 1, wherein the protrusive element is of sufficient dimension to decompress tissues around the pharynx and thereby enable pharyngeal expansion.

11. The intraoral device of claim 1, wherein the protrusive element is of sufficient dimension to stabilize the lateral pharyngeal wall by applying tension to the pterygomandibular raphe, which is coupled to the pharyngeal constrictors.

12. The intraoral device of claim 1, wherein the protrusive element is of sufficient dimension to splay the tonsillar arches formed by the palatoglossus and the palatopharyngeal muscles, leading to further stabilization of the lateral pharyngeal wall.

13. The intraoral device of claim 1, wherein the protrusive element distends between 5 and 15 mm, and optimally 10 mm downwardly from the main body.

14. The intraoral device of claim 1, wherein the dimensions of the protrusive element are adjusted by way of a positioning screw.

15. The intraoral device of claim 1 wherein the dimensions of the protrusive element are adjusted by way of snaps.

16. The intraoral device of claim 1 wherein the dimensions of the protrusive element are adjusted by way of a slidable means.

17. The intraoral device of claim 1, wherein said advancement ranges between 1.0 and 7.0 mm, and preferably commences with a protrusion of 3.0 to 4.0 mm, or approximately ½ of the total protrusive range.

18. A means for treating a user having sleep disorders by way of the employment of an intraoral mandibular advancement device, comprising:
(a) evaluating the patient and selecting a direct or indirect approach, wherein said direct approach includes the prescription of the device combined with its relinement for attachment to the user and said indirect approach includes the taking of an impression of both arches and a bite record of the user followed by prescription of the device in accordance with said impression;
(b) delivery of said device to said patient with instructions on usage and monitoring;
(c) follow up evaluation and adjustment of said device where necessary, wherein:
(d) said device comprises: (1) main body means for attachment to the user's mouth and having a central portion; (2) a protrusive element distending from the central portion of said main body such that when worn by the user said element causes mandibular advancement sufficient to expand the oropharangeal space and reduce the obstruction; and (3) retention means extending from said main body for retention of the device in the user's mouth when worn during the user's sleep state.

19. The device of claim 1, wherein the material composition is selected from the group consisting of polymeric materials, acrylic materials, natural materials, rubbers, metals, silicones, vinyls, elastomeric materials, hard plastic, thermal plastic, and thermosensitive acrylic resin and combinations thereof.
